(19) United States
(12) Patent Application Publication
(10) Pub. No.: US 2020/0093869 A1
(43) Pub. Date: Mar. 26, 2020

(54) LIP-CARE LIPSTICK WITH REPAIR EFFECT

(71) Applicant: Great You Daily Products Co.,Ltd., Guangdong (CN)

(72) Inventor: YUN XING CHAN, Guangdong (CN)

(73) Assignee: Great You Daily Products Co.,Ltd., Guangdong (CN)

(21) Appl. No.: 16/200,669

(22) Filed: Nov. 27, 2018

(30) Foreign Application Priority Data
Aug. 17, 2018 (CN) 201810940973.1

(51) Int. Cl. A61K 35/644; A61K 8/34; A61K 8/49; A61K 8/73; A61K 8/37; A61K 8/9789; A61K 8/92; A61K 8/9794

(52) U.S. Cl. CPC A61K 35/644; A61K 8/345; A61K 8/4946; A61K 8/735; A61K 8/375; A61K 2800/43; A61K 8/9789; A61K 8/927; A61K 8/498; A61K 8/9794; A61K 8/37

(57) ABSTRACT

A lip-care lipstick with a repair effect relates to the technical field of daily necessities. The present invention aims to provide a lip-care lipstick with a repair effect. The lip-care lipstick includes the following components in parts by weight: 3-5 parts by weight of the Manuka honey containing the ME lip-care factor, 3-5 parts by weight of the royal jelly, 0.1-1 part of dipropylene glycol, 0.5-1.5 parts of allantoin, 22 parts by weight of the sodium hyaluronate, 15 parts by weight of polyglyceryl-2 diisostearate, 10 parts by weight of dipentaerythrityl hexahydroxystearate, 10 parts by weight of ethylhexyl palmitate, 8 parts by weight of tridecyl trimellitate, 5 parts by weight of avocado butter, 10 parts by weight of beeswax, 0.9 part by weight of tocopheryl acetate, 0.1 part by weight of methylparaben, 6 parts by weight of colorant and 5 parts by weight of other plant essential oils.

### CROSS-REFERENCE TO RELATED APPLICATION

This application claims the priority benefit of China application serial no. 201810940973.1, filed on Aug. 17, 2018. The entirety of the above-mentioned patent application is hereby incorporated by reference herein and made a part of this specification.

### BACKGROUND OF THE INVENTION

### 1. Field of the Invention

The present invention relates to the technical field of daily necessities, in particular, to a lip-care lipstick with a repair effect.

### 2. Description of Related Art

The skin of the lips is different from the skin of other parts. The skin of the lips has a thin stratum corneum and has no accessory organs such as hair follicles and sebaceous glands. Many salivary glands are distributed in the stratum corneum, and its stratum corneum and stratum granulosum are thin and have less granular melanin than ordinary skin.

Cosmetic lipstick products currently on the market are mainly composed of lanolin, wax and dye. The lanolin has strong absorptivity, and can adsorb dust, bacteria, viruses and some heavy metal ions in the air onto the lip mucosa. The wax such as paraffin, white oil, or ozocerite is derived from petroleum. In addition, the lanolin and the wax have complex ingredients, easily causing allergic reactions such as cracking and peeling of the lip mucosa, and sometimes itching or slight pain in the lips. According to foreign related data, for 18.2% of young female cancer patients, causes of cancer are related to lipstick smearing. The lipstick contains a high amount of heavy metals such as lead and cadmium. Lead is accumulative, and long-term intake to the human body may lead to chronic lead poisoning, causing anemia, abdominal pain, acute kidney failure and other problems. When people drink and eat, the lipstick and these harmful substances thereon can be brought into their mouths, which directly enter the human body.

### SUMMARY OF THE INVENTION

The present invention is firstly directed to a lip-care lipstick with a repair effect. The lip-care lipstick contains a Manuka honey active ingredient ME lip-care factor and sodium hyaluronate, can prevent the lips from cracking and peeling and can promote the metabolism of tender skin.

The present invention further provides a preparation method of the lip-care lipstick, which is simple and convenient for operation. The method can retain effective ingredients of the Manuka honey active ingredient ME lip-care factor and sodium hyaluronate in the lipstick as much as possible; adding plant essential oil at the end can effectively reduce the volatilization of the essential oil; and the prepared lipstick has no granular sensation, and is smoother and more uniform in color when used.

The former technical solution provided by the present invention is as follows: a lip-care lipstick with a repair effect includes the following components in parts by weight: 3-5 parts by weight of Manuka honey containing an ME lip-care factor, 3-5 parts by weight of royal jelly, 0.1-1 part of dipropylene glycol, 0.5-1.5 parts of allantoin, 22 parts by weight of sodium hyaluronate, 15 parts by weight of polyglyceryl-2 diisostearate, 10 parts by weight of dipentaerythrityl hexahydroxystearate, 10 parts by weight of ethylhexyl palmitate, 8 parts by weight of tridecyl trimellitate, 5 parts by weight of avocado butter, 10 parts by weight of beeswax, 0.9 part by weight of tocopheryl acetate, 0.1 part by weight of methylparaben, 6 parts by weight of colorant and 5 parts by weight of other plant essential oils.

The ME lip-care factor (Manuka Essential) refers to a composition of active ingredients such as methylglyoxal, sugar, protein, total phenol, minerals and phenolic compounds naturally present in Manuka honey, which is named the ME lip-care factor.

The plant essential oil is one or more of neroli essential oil, lemon essential oil, cinnamon essential oil, bergamot essential oil, chamomile essential oil, sage essential oil, aloe vera essential oil, jasmine essential oil, lavender essential oil and rose flower oil.

The colorant is one or more of CI 77891, CI 19140, CI 15850, CI 45380, CI16035, CI77491, CI77492 and CI77499.

The latter technical solution provided by the present invention is as follows: a preparation method of the lip-care lipstick with a repair effect includes the following steps:

step 1: accurately weighing the raw materials according to a formula;

step 2: mixing and stirring the polyglyceryl-2 diisostearate, the dipentaerythrityl hexahydroxystearate, the ethylhexyl palmitate and the tridecyl trimellitate with the colorant and the methylparaben uniformly;

step 3: adding the beeswax, mixing uniformly, heating to 70-90° C., and then cooling to 10-50° C.; and

step 4: adding the tocopheryl acetate, the Manuka honey containing the ME lip-care factor, the royal jelly, the dipropylene glycol, the allantoin, the avocado butter and the sodium hyaluronate, mixing uniformly, grinding by a grinder, adding the other plant essential oils, mixing uniformly, and pouring an obtained mixture into a mold for forming.

After the heating to 70-90° C. in step 3, the temperature is maintained constant for 15-25 minutes.

The particle size of ground particles in step 4 is ≤400 nanometers.

The present invention has the following beneficial effects:

The Manuka honey containing the ME lip-care factor is rich in vitamins A, B1, B2, B6 and E, protein and fatty acid, has the functions of nourishing and moisturizing, and plays a role of soothing and resisting allergy, is vegetable oil with excellent skin care and moisturizing effects, is extremely beneficial for lip dryness, lip cracking or discomfort such as lip peeling due to climate change, and is a natural wetting agent. The Manuka honey is rich in minerals, protein and various vitamins, can promote cell growth and collagen synthesis, and is capable of effectively preventing lip allergies and eliminating swelling, dryness, inflammation and the like. Methylglyoxal naturally existing in Manuka honey is a urease inhibitor, which can effectively inhibit the activity of urease, causing bacteria to fail in producing ammonia, and causing bacteria to die in an acidic environment, thereby having an obvious antibacterial effect. The Manuka honey contains a unique Manuka factor (UMF), which has strong and unique antibacterial and anti-oxidation capabilities.

Royal jelly has anti-oxidation, antibacterial and anti-inflammatory effects. It has a strong effect on cell repair and regeneration. Superoxide dismutase (SOD) detected in the royal jelly is a major anti-oxidation component. The royal jelly has an antibacterial effect and an effect of promoting healing of wound infections. A test also shows that the antibacterial spectrum of the royal jelly includes Escherichia coli, Staphylococcus aureus, Salmonella typhi, Streptococcus, Proteus, Bacillus subtilis, Mycobacterium tuberculosis, trichophytosis, epidermophytosis, etc. The antibacterial and anti-inflammatory effects of the royal jelly are related to pH. When the pH is 4.5, the antibacterial effect is the highest; when the pH is 7, the antibacterial effect is weakened; and when the pH is 8, the antibacterial effect disappears. The pH of the royal jelly is 3.5-4.5, so it has the highest antibacterial and anti-inflammatory effects under natural conditions.

Tocopheryl acetate has a strong anti-oxidation effect, delays aging, reduces pigmentation of the lips, protects the lips from loss of moisture, effectively prevents wrinkles of the lips, and makes the lips full and bright red.

The natural plant essential oil added in the invention has a good skin care effect, is capable of decomposing accumulated pigments in the lips, accelerating the balance of nutrients and moisture and enhancing the discharge of cytotoxin, has anti-aging, moisturizing and anti-wrinkle effects, and makes the lip color looks healthier and moisturized. The plant essential oil is the only essential oil with natural care elements that can penetrate deep into the skin and the internal circulation of the human body without leaving chemical residues. It is an aromatic liquid substance that is extracted from flowers, leaves and seeds of natural plants. The molecular structure of the essential oil is very small, so it is more acceptable to the skin, and the absorption rate is almost perfect. The most important thing is that it is natural, pure and has no side effects.

Hyaluronic acid is regarded as the most ideal natural moisturizing factor. Its aqueous solution has strong viscoelasticity and lubricity. It can be applied to the surfaces of the lips to form a moisturizing and breathable layer to keep the lips moist and lustrous. The hyaluronic acid can remove active free radicals generated by ultraviolet radiation in sunlight in the epidermis, protect the lips from injuries and reduce pigmentation; and when used in combination with other nutrients, it can have a more ideal effect to promote nutrient absorption.

### BRIEF DESCRIPTION OF THE DRAWING

FIG. 1 is a graph showing results of a repair performance test.

### DESCRIPTION OF THE EMBODIMENTS

The further detailed description is made for the claims of the present invention below with reference to the specific embodiments, but does not constitute any limitation to the present invention. Any limited modifications made within the protection scope of the claims of the present invention are still within the protection scope of the appended claims.

### Embodiment 1

Step 1: Accurately weigh 3 parts by weight of Manuka honey containing an ME lip-care factor, 5 parts by weight of royal jelly, 1 part of dipropylene glycol, 0.5 part of allantoin, 22 parts by weight of sodium hyaluronate, 15 parts by weight of polyglyceryl-2 diisostearate, 10 parts by weight of dipentaerythrityl hexahydroxystearate, 10 parts by weight of ethylhexyl palmitate, 8 parts by weight of tridecyl trimellitate, 5 parts by weight of avocado butter, 10 parts by weight of beeswax, 0.9 part by weight of tocopheryl acetate, 0.1 part by weight of methylparaben, 6 parts by weight of colorant and 5 parts by weight of other plant essential oils.

Step 2: Mix and stir the polyglyceryl-2 diisostearate, the dipentaerythrityl hexahydroxystearate, the ethylhexyl palmitate and the tridecyl trimellitate with the colorant and the methylparaben uniformly.

Step 3: Add the beeswax, mix uniformly, heat to 90° C., maintain the temperature constant for 15 minutes, and then cool to 50° C.

Step 4: Add the tocopheryl acetate, the Manuka honey containing the ME lip-care factor, the royal jelly, the dipropylene glycol, the allantoin, the avocado butter and the sodium hyaluronate, mix uniformly, grind with a grinder to a particle size of ≤400 nanometers, add the other plant essential oils, mix uniformly, and pour an obtained mixture into a mold for forming.

### Embodiment 2

Step 1: Accurately weigh 5 parts by weight of Manuka honey containing an ME lip-care factor, 3 parts by weight of royal jelly, 0.1 part of dipropylene glycol, 1.5 parts of allantoin, 22 parts by weight of sodium hyaluronate, 15 parts by weight of polyglyceryl-2 diisostearate, 10 parts by weight of dipentaerythrityl hexahydroxystearate, 10 parts by weight of ethylhexyl palmitate, 8 parts by weight of tridecyl trimellitate, 5 parts by weight of avocado butter, 10 parts by weight of beeswax, 0.9 part by weight of tocopheryl acetate, 0.1 part by weight of methylparaben, 6 parts by weight of colorant and 5 parts by weight of other plant essential oils.

Step 2: Mix and stir the polyglyceryl-2 diisostearate, the dipentaerythrityl hexahydroxystearate, the ethylhexyl palmitate and the tridecyl trimellitate with the colorant and the methylparaben uniformly.

Step 3: Add the beeswax, mix uniformly, heat to 70° C., maintain the temperature constant for 25 minutes, and then cool to 10° C.

Step 4: Add the tocopheryl acetate, the Manuka honey containing the ME lip-care factor, the royal jelly, the dipropylene glycol, the allantoin, the avocado butter and the sodium hyaluronate, mix uniformly, grind with a grinder to a particle size of ≤400 nanometers, add the other plant essential oils, mix uniformly, and pour an obtained mixture into a mold for forming.

### Embodiment 3

Step 1: Accurately weigh 4 parts by weight of Manuka honey containing an ME lip-care factor, 4 parts by weight of royal jelly, 0.5 part of dipropylene glycol, 0.9 part of allantoin, 22 parts by weight of sodium hyaluronate, 15 parts by weight of polyglyceryl-2 diisostearate, 10 parts by weight of dipentaerythrityl hexahydroxystearate, 10 parts by weight of ethylhexyl palmitate, 8 parts by weight of tridecyl trimellitate, 5 parts by weight of avocado butter, 10 parts by weight of beeswax, 0.9 part by weight of tocopheryl acetate, 0.1 part by weight of methylparaben, 6 parts by weight of colorant and 5 parts by weight of other plant essential oils.

Step 2: Mix and stir the polyglyceryl-2 diisostearate, the dipentaerythrityl hexahydroxystearate, the ethylhexyl palmitate and the tridecyl trimellitate with the colorant and the methylparaben uniformly.

Step 3: Add the beeswax, mix uniformly, heat to 80° C., maintain the temperature constant for 20 minutes, and then cool to 35° C.

Step 4: Add the tocopheryl acetate, the Manuka honey containing the ME lip-care factor, the royal jelly, the dipropylene glycol, allantoin, the avocado butter and the sodium hyaluronate, mix uniformly, grind with a grinder to a particle size of ≤400 nanometers, add the other plant essential oils, mix uniformly, and pour the obtained mixture into a mold for forming.

1 g of a lip-care lipstick prepared in Embodiment 1 was weighed, added into 9 ml of pure water, mixed and ground for 5 min, and supernatant was removed by centrifugation to prepare 10 mg/ml and 50 mg/ml test samples. The test samples passed through a microfiltration membrane to obtain sterile test sample solutions, which were placed in a refrigerator at 20° C. for later use. The sterile test sample solutions and a control group (without adding the lip-care lipstick prepared in Embodiment 1) were respectively tested for repair performance and moisturizing performance.

1. Repair Performance Test

HaCaT cell single molecules were inoculated in 10 mg/ml and 50 mg/ml sterile test sample solutions and cultured for 19 h, and a central interstitial region was observed by microscopic imaging analysis. The test was repeated for 3 times, and results were averaged.

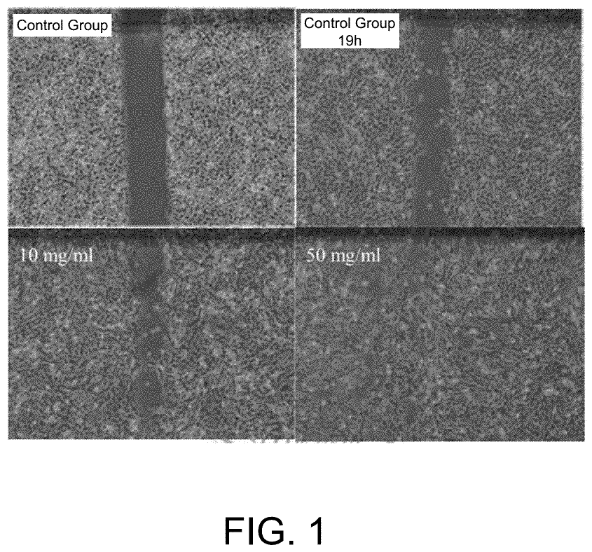

The detailed results are shown in Table 1 and FIG. 1:

TABLE 1
Integral area of HaCaT cell migration repair capacity
in determination of repair performance (cm²)

| Group | Average (uM) | Standard Deviation |
|---|---|---|
| Control group | 13.1 | 2.5 |
| LPS | 9.3 | 1.7 |
| 10 mg/ml | 4.7 | 1.5 |
| 50 mg/ml | 1.8 | 1.2 |

The test proved that a blank region of the control group was significantly reduced by 38%, the blank region was significantly reduced by 70% when the 10 mg/ml sterile test sample solution was added, and the blank region was significantly reduced by 88% when the 50 mg/ml sterile test sample solution was added, indicating that the lip-care lipstick prepared in Embodiment 1 has a significant repair effect.

2. Moisturizing Performance Test

The lip-care lipstick obtained in Embodiment 1 was applied to the left half of lips of a testee, and the right half of lips was used as a control group for comparison. After 15 min, the lips were cleaned with water, and gently touched with lens wiping paper. After 5 min, the moisture and skin elasticity were tested with an SG-S TSII skin multifunction detector.

Detailed results are shown in Table 2.

TABLE 2
Results of moisturizing performance test

| Moisturizing Test Data | | | Elasticity Test Data | | |
|---|---|---|---|---|---|
| | Control | Lipstick | | Control | Lipstick |
| Testee | Group | Group | Testee | Group | Group |
| 1 | 51 | 63 | 1 | 51 | 57 |
| 2 | 55 | 54 | 2 | 40 | 42 |
| 3 | 58 | 56 | 3 | 45 | 59 |
| 4 | 42 | 51 | 4 | 40 | 49 |
| 5 | 46 | 53 | 5 | 30 | 30 |
| 6 | 58 | 54 | 6 | 30 | 39 |
| 7 | 56 | 55 | 7 | 55 | 62 |
| 8 | 49 | 56 | 8 | 48 | 52 |
| 9 | 44 | 49 | 9 | 55 | 51 |
| 10 | 48 | 62 | 10 | 37 | 35 |
| Average | 50.7 ± 5.8 | 56.3 ± 5.1 | Average | 43.1 ± 9.3 | 47.6 ± 10.7 |

The test showed that after the lip-care lipstick obtained in Embodiment 1 was used, the moisture of the lips was increased by at least 11%, and the elasticity of the lip epidermis was increased by at least 10.4%.

The above are only exemplary embodiments of the present invention. Any modifications, equivalent substitutions, improvements and the like made within the spirit and scope of the present invention shall be included within the protection scope of the present invention.

What is claimed is:

1. A lip-care lipstick with a repair effect, comprising the following components in parts by weight: 3-5 parts by weight of Manuka honey containing an ME lip-care factor, 3-5 parts by weight of royal jelly, 0.1-1 part by weight of dipropylene glycol, 0.5-1.5 parts by weight of allantoin, 22 parts by weight of sodium hyaluronate, 15 parts by weight of polyglyceryl-2 diisostearate, 10 parts by weight of dipentaerythrityl hexahydroxystearate, 10 parts by weight of ethylhexyl palmitate, 8 parts by weight of tridecyl trimellitate, 5 parts by weight of avocado butter, 10 parts by weight of beeswax, 0.9 part by weight of tocopheryl acetate, 0.1 part by weight of methylparaben, 6 parts by weight of colorant and 5 parts by weight of plant essential oils.

2. The lip-care lipstick with the repair effect according to claim 1, wherein the plant essential oil is one or more of neroli essential oil, lemon essential oil, cinnamon essential oil, bergamot essential oil, chamomile essential oil, sage essential oil, aloe vera essential oil, jasmine essential oil, lavender essential oil and rose flower oil.

3. The lip-care lipstick with the repair effect according to claim 1, wherein the colorant is one or more of CI 77891, CI 19140, CI 15850, CI 45380, CI16035, CI77491, CI77492 and CI77499.

4. A preparation method of the lip-care lipstick with the repair effect according to claim 1, comprising the following steps:
step 1: accurately weighing raw materials according to a formula;
step 2: mixing and stirring the polyglyceryl-2 diisostearate, the dipentaerythrityl hexahydroxystearate, the ethylhexyl palmitate and the tridecyl trimellitate with the colorant and the methylparaben uniformly;
step 3: adding the beeswax, mixing uniformly, heating to 70-90° C., and then cooling to 10-50° C.; and
step 4: adding the tocopheryl acetate, the Manuka honey containing the ME lip-care factor, the royal jelly, the dipropylene glycol, the allantoin, the avocado butter and the sodium hyaluronate, mixing uniformly, grinding by a grinder, adding the plant essential oils, mixing uniformly, and pouring an obtained mixture into a mold for forming.

5. The preparation method of the lip-care lipstick with the repair effect according to claim 4, wherein after the heating to 70-90° C. in the step 3, the temperature is maintained constant for 15-25 minutes.

6. The preparation method of the lip-care lipstick with the repair effect according to claim 4, wherein the particle size of ground particles in step 4 is ≤400 nanometers.

7. A preparation method of the lip-care lipstick with the repair effect according to claim 2, comprising the following steps:
step 1: accurately weighing raw materials according to a formula;
step 2: mixing and stirring the polyglyceryl-2 diisostearate, the dipentaerythrityl hexahydroxystearate, the ethylhexyl palmitate and the tridecyl trimellitate with the colorant and the methylparaben uniformly;
step 3: adding the beeswax, mixing uniformly, heating to 70-90° C., and then cooling to 10-50° C.; and
step 4: adding the tocopheryl acetate, the Manuka honey containing the ME lip-care factor, the royal jelly, the dipropylene glycol, the allantoin, the avocado butter and the sodium hyaluronate, mixing uniformly, grinding by a grinder, adding the plant essential oils, mixing uniformly, and pouring an obtained mixture into a mold for forming.

8. The preparation method of the lip-care lipstick with the repair effect according to claim 7, wherein after the heating to 70-90° C. in the step 3, the temperature is maintained constant for 15-25 minutes.

9. The preparation method of the lip-care lipstick with the repair effect according to claim 7, wherein the particle size of ground particles in step 4 is ≤400 nanometers.

10. A preparation method of the lip-care lipstick with the repair effect according to claim 3, comprising the following steps:
step 1: accurately weighing raw materials according to a formula;
step 2: mixing and stirring the polyglyceryl-2 diisostearate, the dipentaerythrityl hexahydroxystearate, the ethylhexyl palmitate and the tridecyl trimellitate with the colorant and the methylparaben uniformly;
step 3: adding the beeswax, mixing uniformly, heating to 70-90° C., and then cooling to 10-50° C.; and
step 4: adding the tocopheryl acetate, the Manuka honey containing the ME lip-care factor, the royal jelly, the dipropylene glycol, the allantoin, the avocado butter and the sodium hyaluronate, mixing uniformly, grinding by a grinder, adding the plant essential oils, mixing uniformly, and pouring an obtained mixture into a mold for forming.

11. The preparation method of the lip-care lipstick with the repair effect according to claim 10, wherein after the heating to 70-90° C. in the step 3, the temperature is maintained constant for 15-25 minutes.

12. The preparation method of the lip-care lipstick with the repair effect according to claim 10, wherein the particle size of ground particles in step 4 is ≤400 nanometers.
